(19) United States
(12) Patent Application Publication
(10) Pub. No.: US 2021/0199547 A1
(43) Pub. Date: Jul. 1, 2021

(54) METHODS AND DEVICES FOR HYDROGEL- AND AEROGEL-BASED SAMPLE PRETREATMENT

(71) Applicant: University of Cincinnati, Cincinnati, OH (US)

(72) Inventors: Eliot Gomez, Cincinnati, OH (US); Andrew Jajack, North Canton, OH (US); Amy Drexelius, Covington, KY (US)

(21) Appl. No.: 15/734,340

(22) PCT Filed: Jun. 7, 2019
(86) PCT No.: PCT/US2019/035999

§ 371 Date: Dec. 2, 2020

Related U.S. Application Data
(60) Provisional application No. 62/681,895, filed on Jun. 7, 2018.

(51) Int. Cl. G01N 1/40; G01N 33/543

(52) U.S. Cl. CPC G01N 1/405; G01N 33/54313; G01N 2001/2826; G01N 1/4077; G01N 33/5436

(57) ABSTRACT

Hydrogel-based or aerogel-based devices and methods for pretreatment of a sample. The devices and method may include an aerogel-based device for pretreating a sample comprising at least one aerogel; a hydrogel-based device for pretreating a sample comprising at least one hydrogel; or a combination aerogel-based and hydrogel-based device for pretreating a sample comprising at least one aerogel in fluid communication with at least one hydrogel.

### CROSS-REFERENCE TO RELATED APPLICATIONS

This application claims priority to and the benefit of the filing date of U.S. Provisional Patent Application Ser. No. 62/681,895, entitled “Methods and Devices for Hydrogel- and Aerogel-Based Sample Pretreatment,” filed Jun. 7, 2018, the disclosure of which is incorporated by reference herein in its entirety.

### STATEMENT REGARDING FEDERALLY SPONSORED RESEARCH

This invention was made with government support under Grant No. 1013160 awarded by the Ohio Federal Research Network. The government has certain rights in the invention.

### FIELD OF THE INVENTION

The present invention relates generally to the field of immunodiagnostic assays, and more specifically to devices and methods for addressing sensitivity limitations of present immunodiagnostic assays.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Immunodiagnostic assays include biochemical tests that report or measure the presence or concentration of a macromolecule or a small molecule in a sample. Many immunodiagnostic assays typically use antibodies and gold conjugates or florescent tags to indicate the presence of a target antigens (the molecule of interest/analyte of interest). A common type of immunodiagnostic assay utilizes a lateral flow assay device, such as the pregnancy test strip, as well as other lateral flow assays devices to detect for diseases (Legionella, influenza, C. Difficile, etc.). However, sensitivity is a problem for immunodiagnostics, as well as other rapid diagnostic tests (enzymatic or aptamer-based sensors), since the sample fluid is often dilute and thus the concentration of the analyte of interest may fall below the limit of detection of these devices.

These sensitivity limitations may be overcome by pretreating the sample to increase the antigen concentration while simultaneously decreasing the amount of interferents, such as large molecules like proteins (e.g. mucins, serum, etc.), small molecules (e.g. salt, etc.), and other interferents (e.g., pH). Such sample pretreatment is typically done with conventional laboratory processes (e.g. centrifugation, buffering, lipid scrubbing, pH, etc.) that require multi-step processes with equipment that is not compatible with a rapid and portable test format. Portable sample pretreatment for rapid diagnostics depends on developing processes that are automatic and passively driven with as few steps as possible

Most sample pretreatment can be done through a series of filtration membranes, which is broadly illustrated in the device 10 shown in FIG. 1, that effectively create a bandpass filter for a narrow range of molecule sizes. FIG. 1 shows a sample 12 entering a device 10 (which could be driven with pressure, gravity, or capillary action). The sample contains the analyte 14 to be detected (diamonds, e.g., 50 kDa), and interferents such as large molecules 16 (large circles, e.g., >100 kDa) and small molecules 18 (small circles, e.g., <10 kDa). A first membrane 20 contains 100 kDa pores that removes only the large molecules 16. A second membrane 22 (10 kDa) concentrates the sample using (as an example) forward osmosis to remove water. The small molecules 18 are not rejected by the second membrane 22 and thus passively diffuse through the second membrane 22. As a result, the analyte 14 is concentrated before it proceeds to a sensor.

Devices and methods of sample pretreatment that can operate according to principles similar to those shown in FIG. 1, while allowing for a rapid and portable test format, are desirable.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Various aspects of the present invention overcome the drawbacks described above in the Background of the Invention section. These aspects do so by providing hydrogel-based or aerogel-based devices and methods for pretreatment of a sample. The devices may include an aerogel-based device for pretreating a sample comprising at least one aerogel; a hydrogel-based device for pretreating a sample comprising at least one hydrogel; or a combination aerogel-based and hydrogel-based device for pretreating a sample comprising at least one aerogel in fluid communication with at least one hydrogel. The methods of pretreating a sample may include contacting a fluid sample with an aerogel or a hydrogel, wherein said aerogel or said hydrogel includes (i) a filter layer, and (ii) a fluid storage layer.

Additional aspects of the present invention may include methods of making devices for pretreating a sample. Such methods may include positioning a first hydrogel and a second hydrogel adjacent to one another, wherein the density of the first hydrogel is different from the density of the second hydrogel; and removing water from the first hydrogel and the second hydrogel to form an aerogel, said aerogel including a first layer and a second layer.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a schematic of a two stage membrane that provides a bandpass filter of analyte sizes.

FIGS. 2A and 2B are schematics showing a hydrogel freeze-dried to form an aerogel used as a membrane and wick.

FIGS. 3A and 3B are schematics showing freeze-dried hydrogel (aerogel) beads that remove water and reject analyte.

FIGS. 4A and 4B are schematics showing hydrogel actuators that contract to pretreat a sample.

FIG. 5 is a schematic showing band pass membranes with hydrogel interaction.

### DEFINITIONS

“Aerogel,” as used herein, means a porous polymer or synthetic matrix derived from a gel (e.g., hydrogel) wherein the liquid has been replaced with a gas.

“Sample pretreatment,” as used herein, means processing done to a sample of fluid to concentrate (e.g., concentrate an analyte of interest), add reagents, buffer, or remove interferents.

“Immunodiagnostic assays,” as used herein, means biochemical tests that report or measure the presence or concentration of a macromolecule or a small molecule in a solution, as may be done through the use of an antibody or an antigen.

“Rapid diagnostic test,” as used herein, means a medical diagnostic test that is quick and easy to perform, (also known as point-of-care). It may include immunodiagnostic and other enzymatic sensors (e.g. glucose).

“Membrane,” as used herein, means a selective barrier that acts as a boundary for molecules, ions, proteins, or other small particles. Membranes may be size selective or charge selective.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Various aspects of the present invention overcome the drawbacks described above in the Background of the Invention section. As described in the Background, presently sample pretreatment (such as filtration to concentrate an analyte of interest) is typically done with conventional laboratory processes that require multi-step processes with equipment that is not compatible with a rapid and portable test format. For example, the driving mechanism of membrane filtration typically requires a pump, which is not amenable to rapid diagnostics. Embodiments of the present invention, however, are based on the use of hydrogels, or hydrogels with the water removed (also known as an aerogels), as a wick, membrane, and/or device containing antibodies to an analyte of interest.

Hydrogels include a network of polymers or synthetic materials that are highly absorbent and contain a substantial amount of water (e.g., over 90% water). Some examples of hydrogels are agarose, sodium polyacrylate, poly(vinyl alcohol), Poly(ethylene glycol), etc., but they can also be synthetic materials (e.g. silica, carbon, metal oxide). The density of hydrogels can be controlled by increasing the concentration of the material (in the case of agarose) or by increasing the crosslink agent that creates the network. The density of hydrogel is frequently used in molecular biology for the separation of molecules including DNA electrophoresis and protein purification.

One aspect of the present invention involves the removal of water from a hydrogel using freeze-dried or solvent exchange techniques while the integrity of the polymeric structure remains to form an aerogel. The aerogel then can act simultaneously as a wick and size-exclusion membrane when exposed to the sample. For example, an agarose (2 wt %) typically contains pore sizes ranging from 100-200 nm. If the agarose is freeze-dried and the structure retained, it will readily absorb water while filtering out particles larger than 200 nm. The aerogel thus acts both as a membrane and a driving wick. An embodiment in accordance with this aspect of the present invention is illustrated in FIGS. 2A and 2B where a sample 24 is brought into contact with an aerogel 26 containing first and second layers 28, 30. The first and second layers 28, 30 may be fabricated by positioning hydrogels of different density on top of each other and freeze-drying the layers. The first layer 28 is a dense polymeric network that rejects the analyte of interest. The second layer 30 is a fluid storage layer. The fluid storage layer 30 may have a set capacity of volume. When the fluid sample 24 is brought into contact with the aerogel, the aerogel will pull fluid 34 (e.g., water from the sample 24) into the fluid storage layer until the volume reaches fluid capacity. Fluid wicks into the reservoir resulting in the analyte 32 of interest being concentrated on the outside of the aerogel structure (because the first layer 28 rejects the analyte 32). Once the sample is pretreated in this manner, the sample on the outside of the aerogel structure (including the now-concentrated analyte of interest) may be further processed, such as by being dispensed onto a sensor.

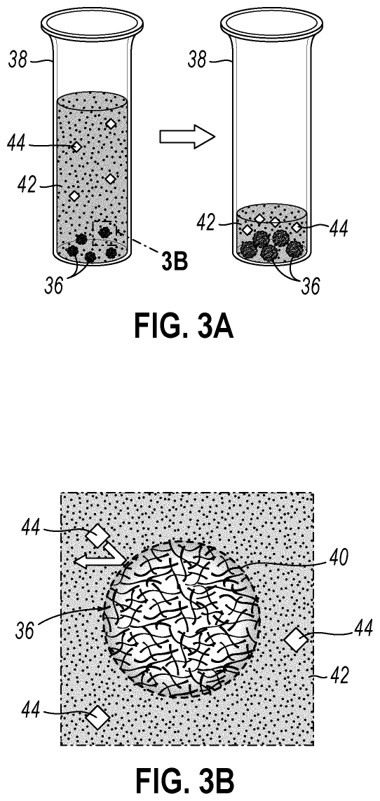

Referring now to FIGS. 3A and 3B, another embodiment of the present invention is directed to aerogel beads 36, which function similarly to the embodiment illustrated in FIGS. 2A and 2B. The aerogel beads may be placed in a vial 38. The aerogel includes a membrane 40 that is provided by either the pores of the aerogel or by being attached to a separate membrane that rejects the analyte 44. The sample 42 is added to the vial and the aerogel draws in fluid (e.g., water). The beads can then be removed from the vial and the concentrated analyte remains in the bulk solution in the vial.

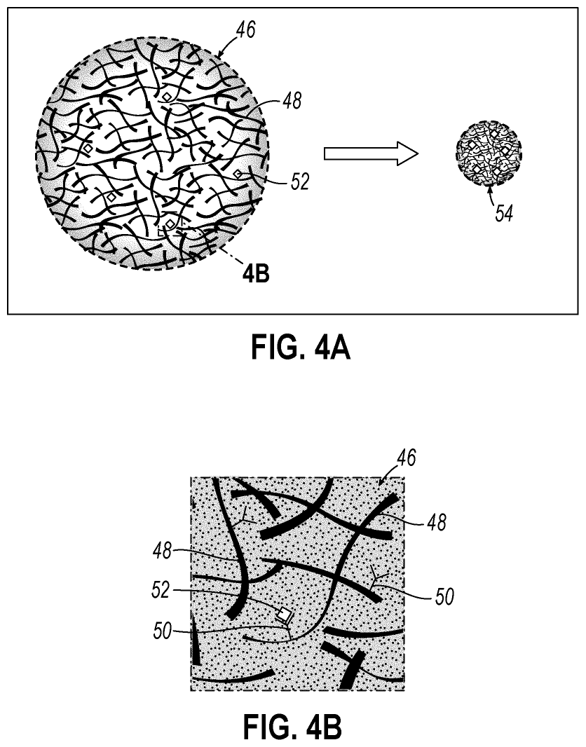

Referring now to FIGS. 4A and 4B, another embodiment of the present invention is shown that uses a hydrogel as a component of the device for pretreating a sample. As is known, hydrogels can change properties when exposed to external stimuli; such hydrogels are often referred to as “smart gels”. Changes in pH, temperature, ionic concentration, or application of an electric field can cause some hydrogels to change shape or release a ligand, and have been used for drug delivery systems.

To leverage the properties of such “smart” hydrogels, the embodiment shown in FIGS. 4A and 4B uses a hydrogel 46 having sensing probes or other sensing modalities (enzymes, aptamers, etc.) associated with the polymer matrix 48, such as by being covalently bound or trapped in the matrix itself. Such modalities may include one or more antibodies 50 to an antigen 52 of interest. The hydrogel is then converted to an aerogel. The density of the hydrogel is chosen in this embodiment such that the outer layer of the resulting aerogel will allow passage of the antigen of interest. When a sample fluid then is brought into contact with the aerogel, the sample fluid rehydrates the aerogel matrix into a hydrogel again, with the antigen of interest entering the matrix and binding to the antibodies. FIGS. 4A and 4B show an embodiment of a hydrogel that contains bound antibodies to its polymer matrix.

Once the fluid sample has contacted and rehydrated the aerogel, an external stimulus is applied (e.g. pH change or ionic concentration) that causes the hydrogel to contract 54. When this occurs, the hydrogel pores reduce in size during the dynamic shift, causing the analytes to be rejected and remain inside of the matrix. The solution in the hydrogel thus becomes concentrated. And the hydrogel beads can be read directly using a reporter or used for further processing.

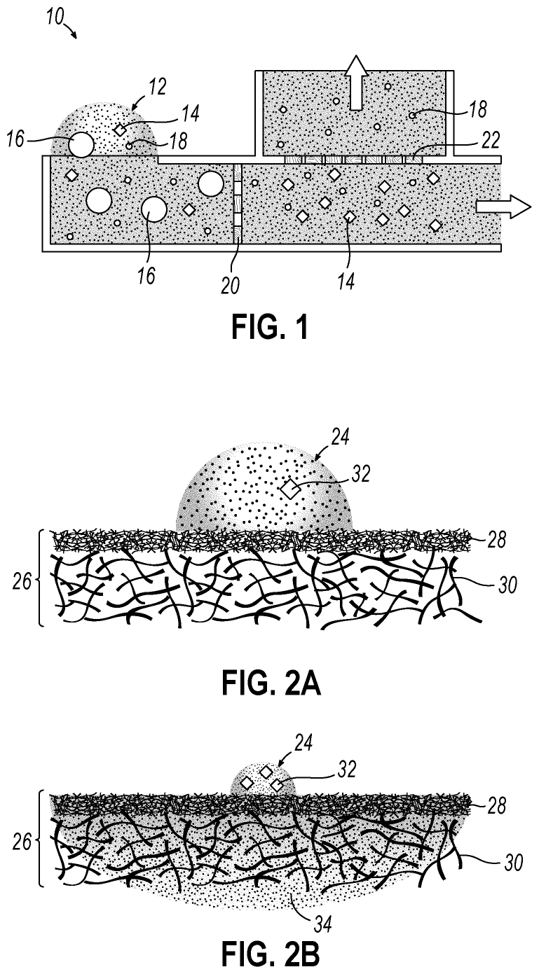

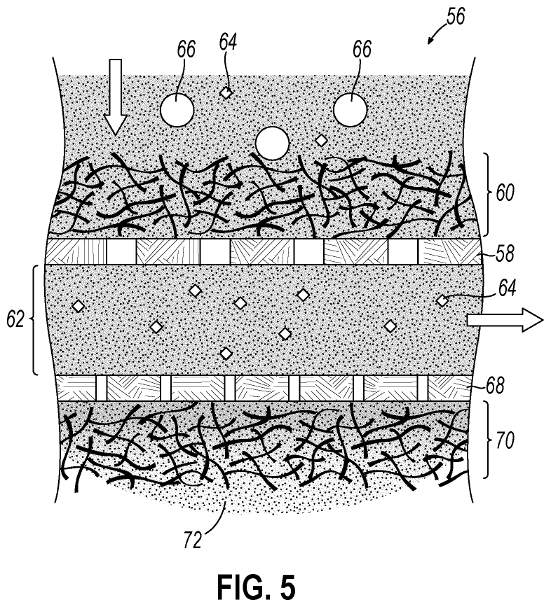

Referring now to FIG. 5, another embodiment of the present invention is shown. This embodiment combines the different layers of hydrogel/aerogel structures to form a molecular bandpass filter 56 (shown in FIG. 5) that corresponds to the principles shown in FIG. 1. The device in this embodiment is created by preparing hydrogels with different densities, positioning the hydrogels relative to one another, and freeze-drying the hydrogels to create an aerogel. Upon adding the fluid sample to the device, the sample wicks into the subsequent layers. In FIG. 5, the first two layers 58, 60 remove large molecules 66 and prevent fouling. The analyte 64 then proceeds to an inner channel 62 where it is rejected by a 10 kDa layer 68. The water wicking reservoir 70 pulls water 72 and other small molecules (<10 kDa) past the 10 kDa layer until the volume of the reservoir is full. The inner channel may be either an open channel or another wicking material that carries the fluid therein (i.e., the collected and concentrated analyte in the inner channel) to the next stage.

The embodiments of the present invention recited herein are intended to be merely exemplary and those skilled in the art will be able to make numerous variations and modifications to it without departing from the spirit of the present invention. Notwithstanding the above, certain variations and modifications, while producing less than optimal results, may still produce satisfactory results. All such variations and modifications are intended to be within the scope of the present invention as defined by the claims appended hereto.

What is claimed is:

1. A device for concentrating an analyte of interest, comprising:
an aerogel, said aerogel including (i) a filter layer, and (ii) a fluid storage layer.

2. The device of claim 1, wherein the aerogel is in the form of a bead.

3. The device of claim 2, further comprising a plurality of beads, each of said beads including said aerogel.

4. The device of claim 2, wherein said bead is associated with a container, and is disposed within an interior space of said container.

5. The device of claim 1, wherein said aerogel includes a polymer or synthetic matrix.

6. The device of claim 5, further comprising one or more antibodies bound to said polymer or synthetic matrix.

7. The device of claim 6, wherein said one or more antibodies have the capability to bind the analyte of interest.

8. The device of claim 1, wherein said aerogel further comprises a second filter layer.

9. The device of claim 8, wherein said first filter layer allows the passage of larger molecules than said second filter layer.

10. The device of claim 9, wherein said first filter layer allows the passage of molecules up to 100 kDa, and wherein said second filter layer allows the passage of molecules up to 10 kDa.

11. A device for concentrating an analyte of interest, comprising:
a hydrogel, said hydrogel including (i) a filter layer, (ii) a fluid storage layer, and (iii) one or more sensing modalities present in a polymer or synthetic matrix of the fluid storage layer.

12. The device of claim 11, wherein said one or more sensing modalities include one or more antibodies to the analyte of interest.

13. The device of claim 11, wherein the hydrogel is adapted to contract upon the application of an external stimulus.

14. The device of claim 13, wherein said external stimulus is selected from pH change, temperature, ionic concentration, and application of an electric field.

15. A device for pretreating a sample, comprising:
at least one aerogel, said at least one aerogel including (i) a filter layer, and (ii) a fluid storage layer.

16. The device of claim 15, wherein pretreating a sample is selected from concentrating a component in the sample, adding a reagent to the sample, buffering the sample, removing interferents from the sample, and combinations thereof.

17. The device of claim 15, further comprising at least one hydrogel, wherein said at least one aerogel is in fluid communication with said at least one hydrogel.

18. A method of making a device for concentrating an analyte of interest, the method comprising:
positioning a first hydrogel and a second hydrogel adjacent to one another, wherein the density of the first hydrogel is different from the density of the second hydrogel; and
removing water from the first hydrogel and the second hydrogel to form an aerogel, said aerogel including a first layer and a second layer.

19. The method of claim 18, wherein water is removed from the first hydrogel and the second hydrogel via a process selected from freeze-drying and solvent exchange.

20. The method of claim 18, further comprising introducing one or more antibodies into a polymer matrix of the first hydrogel, the second hydrogel, or the first and second hydrogels prior to removing water from the first hydrogel and the second hydrogel.

21. A method of pretreating a sample, the method comprising:
contacting a fluid sample with an aerogel or a hydrogel, wherein said aerogel or said hydrogel includes (i) a filter layer, and (ii) a fluid storage layer.

22. The method of claim 21, wherein said fluid sample is contacted with a hydrogel, and the method further comprising applying an external stimulus to said hydrogel to cause said hydrogel to contract.

23. The method of claim 22, wherein said external stimulus is selected from pH change, temperature, ionic concentration, and application of an electric field.

24. The method of claim 21, wherein said fluid sample is contacted with an aerogel, and the method further comprising further comprising contacting a sensor with said fluid sample, after said fluid sample has contacted said aerogel.
